(19) United States
(12) Patent Application Publication
(10) Pub. No.: US 2022/0143411 A1
(43) Pub. Date: May 12, 2022

(54) SYSTEMS AND METHODS TO REDUCE RF-INDUCED HEATING OF AN IMPLANTED LEAD

(71) Applicant: Advanced Neuromodulation Systems, Inc., Plano, TX (US)

(72) Inventors: Jeffrey Urbanski, Frisco, TX (US); Ted Alfonso, Frisco, TX (US); John Gonzalez, Collin, TX (US)

(21) Appl. No.: 17/091,336

(22) Filed: Nov. 6, 2020

(51) Int. Cl. A61N 1/375; A61N 1/36

(52) U.S. Cl. CPC A61N 1/375; A61N 1/36175; A61N 1/36067; A61N 1/36071; A61N 1/36125; A61N 1/36062

(57) ABSTRACT

The present disclosure provides systems and methods for a conductor assembly for an implantable lead cable. The conductor assembly includes a conductive element extending over an axial length from a proximal end to a distal end. The conductor assembly includes an inner dielectric layer coaxially covering the conductive element over the axial length. The conductor assembly includes an inner conductive layer coaxially covering the inner dielectric layer over the axial length, the inner conductive layer comprising a contiguous metal coating having a thickness in a range of 1 to 50 microns.

### A. FIELD OF THE DISCLOSURE

The present disclosure relates generally to neurostimulation systems and, more particularly, to a multi-conductor cable for an implantable lead that reduces RF-induced heating along the cable and at the lead.

### B. BACKGROUND

Neurostimulation is an established neuromodulation therapy for the treatment of chronic pain and movement disorders. Types of neurostimulation include deep brain stimulation (DBS) to treat cardinal motor symptoms of Parkinson's Disease (PD), such as bradykinesia, rigidity, and tremors; spinal cord stimulation (SCS) for treating chronic pain such as Failed Back Surgery Syndrome (FBSS) and Complex Regional Pain Syndrome (CRPS); and Dorsal Root Ganglion (DRG) stimulation for treating CRPS, post-surgical pain, diabetic neuropathy, and other peripheral nerve pain.

Neurostimulation systems typically include one or more implantable leads connected to a pulse generator and electrical power source. The pulse generator may be external (i.e., an external pulse generator, or EPG) or may itself be implantable (i.e., an implantable pulse generator, or IPG) and is controlled via a percutaneous wired connection or wirelessly by a controller. Implantable leads typically include multiple electrodes positioned on a distal structure, or substrate, that is linear, cylindrical, paddle-shaped, or otherwise at least partially conformal to the shape of the target region, which may vary among DBS, SCS, and DRG indications. At least some implantable leads incorporate multiple individually controlled channels (e.g., 4-channel or 8-channel) to enable greater degrees of control in applying neurostimulation.

Implantable leads also include a lead wire, or cable, that connects the distal end of the lead, e.g., the electrodes placed at the target region, to the pulse generator. The cable typically includes multiple electrical conductors, or conductive elements, for delivering electric current, i.e., the therapeutic signal, over one or more channels to the electrodes. Likewise, implantable leads may also be used to sense electrical signals at the target region and deliver those sensed electrical signals to the pulse generator or other receiver or sensing device. Implantable leads are also used in various other long term implant applications, such as, for example, an implanted pacemaker device. Because the lead is implanted permanently, the lead is exposed, from time to time, to electromagnetic fields generated, for example, during magnetic resonance imaging (MRI) procedures. These electromagnetic fields can induce radio frequency (RF) current conducting through the conductors within the lead toward the pulse generator or toward the distal electrodes, and, consequently can result in RF-induced heating along the conductive elements. The magnitude and distribution of the RF-induced heating along the conductive elements weighs on the ability to minimize permanent tissue damage that may result from exceeding temperatures surrounding tissue can tolerate. The low impedance that generally characterizes the implantable lead, including the conductive elements, does not sufficiently impede RF-induced currents from conducting the length of the conductive elements and reaching the proximal or distal ends of the lead. At the proximal end of the implantable lead, RF-induced current is received at the pulse generator or sensing device and is dissipated into a protection circuit to prevent the current reaching more sensitive circuits. At the distal end of the implantable lead, RF-induced current is directed to electrode or sensing surfaces exposed to tissue, thereby increasing the temperature at the tissue interface. The amplitude of the RF-induced current and the duration of exposure to the RF energy dictate the degree of temperature increase.

Accordingly, it is desirable to prevent or at least reduce the RF-induced current conduction, and therefore the RF-induced heating in implantable leads.

### BRIEF SUMMARY OF THE DISCLOSURE

In one embodiment, the present disclosure is directed to a conductor assembly for an implantable lead cable. The conductor assembly includes a conductive element extending over an axial length from a proximal end to a distal end. The conductor assembly includes an inner dielectric layer coaxially covering the conductive element over the axial length. The conductor assembly includes an inner conductive layer coaxially covering the inner dielectric layer over the axial length, the inner conductive layer comprising a contiguous metal coating having a thickness in a range of 1 to 50 microns.

In another embodiment, the present disclosure is directed to a method of fabricating a conductor assembly for an implantable lead cable. The method includes covering a conductive element extending an axial length from a distal end to a proximal end with an inner dielectric layer extending coaxially over the axial length. The method includes applying an inner conductive layer coaxially covering the inner dielectric layer over the axial length, the inner conductive layer comprising a contiguous metal coating having a thickness in a range of 1 to 50 microns.

In another embodiment, the present disclosure is directed to an implantable lead. The implantable lead includes at least one electrode disposed at a distal end opposite a proximal end configured to be coupled to a pulse generator. The implantable lead includes a plurality of conductor assemblies extending over an axial length from the proximal end to the at least one electrode at the distal end. Each conductor assembly includes a conductive element extending over the axial length, an inner dielectric layer coaxially covering the conductive element over the axial length, and an inner conductive layer coaxially covering the inner dielectric layer over the axial length, the inner conductive layer comprising a contiguous metal coating having a thickness in a range of 1 to 50 microns. The implantable lead includes a polymer jacket coaxially covering the plurality of conductor assemblies over the axial length, the polymer jacket electrically isolating respective inner conductive layers of the plurality of conductor assemblies.

The foregoing and other aspects, features, details, utilities and advantages of the present disclosure will be apparent from reading the following description and claims, and from reviewing the accompanying drawings.

### BRIEF DESCRIPTION OF THE DRAWINGS

FIG. 1 is a schematic view of an example neurostimulation system;

FIG. 2 is a cross-section view of an example conductor assembly for an implantable lead;

FIG. 3 is a perspective view of the conductor assembly shown in FIG. 2;

FIG. 4 is a perspective view of the conductor assembly shown in FIGS. 2-3, and including multiple segments of an inner conductive layer;

FIG. 5 is a perspective view of the conductor assembly shown in FIG. 4, and including multiple segments of the inner conductive layer and the outer conductive layer;

FIG. 6 is an illustration of fabrication of one embodiment of a conductor assembly;

FIG. 7 is an illustration of fabrication of one embodiment of a conductor assembly having a conductive shield having a varying resistance over the length of conductor assembly;

FIG. 8 is an illustration of fabrication of one embodiment of a conductor assembly having a segmented conductive shielding;

FIG. 9 is an illustration of another example method of fabrication of one embodiment of a conductor assembly having a segmented conductive shielding;

FIG. 10 is a flow diagram of a method of fabricating the conductor assembly shown in FIGS. 2-6 and 9;

FIG. 11 is a flow diagram of a method of fabricating the conductor assembly shown in FIGS. 2-5 and 8;

FIG. 12 is a flow diagram of a method of fabricating the conductor assembly shown in FIGS. 2-5 and 7; and

FIG. 13 is a perspective view of an implantable lead body having a plurality of the conductor assemblies shown in FIGS. 2-5.

Corresponding reference characters indicate corresponding parts throughout the several views of the drawings.

### DETAILED DESCRIPTION OF THE DISCLOSURE

The present disclosure provides an implantable lead for neurostimulation systems with a multi-conductor cable that reduces RF-induced heating of the implantable lead when exposed, for example, to an electromagnetic field generated during an Mill. The disclosed implantable lead includes conductive elements within the cable that are individually shielded in a manner that minimizes the functional and dimensional impacts of such shielding. Each conductive element is coated in a thin layer of dielectric material and then further fabricated using material deposition to create a conductive skin around the conductive element and its dielectric coating to shield it from RF energy. The shielded conductive elements are then distributed within a polymer tube in a manner that provides isolation between the conductive skin surrounding each conductive element forming the implantable lead and, more specifically, the cable. The electrical isolation, in certain embodiments, is provided by inserting each element into an individual lumen in a polymer tube or by coating each element with a dielectric material. The multi lumen polymer tube provides more consistent control of the element spacing relative to each other, providing more consistent, or at least partially controlled, RF energy dissipation. Alternatively, the conductive skin can be deposited on the outer surface of the polymer tube.

The disclosed conductive skin interacts with the electromagnetic field and directs induced current flows along the conductive skin. The material selection and thickness of the conductive skin can be tuned to provide a desired level of RF shielding for a given implantable lead for a given application, e.g., within the field of neurostimulation or other implantable device. Likewise, the conductive skin may include one or more layers and, accordingly, a dielectric gap between each layer that can be further tuned. The conductive skin may include features that operate to distribute, minimize, reduce, or cancel RF-induced currents along the length of the lead. For example, the features may include constructing the conductive surface as a series of discrete rings having axial lengths that minimize coupling of RF energy and a dielectric gap between each ring that is as narrow as possible based on manufacturing capabilities. Such features localize RF-induced current to each individual conductive surface segment, or ring, and thus would distribute the RF-induced heat generation along the length of the implantable lead. The axial length of the discrete rings and corresponding number of dielectric gaps between each discrete ring may also be tuned to provide the desired RF shielding and heat distribution.

The disclosed conductive skin can be formed by deposition over the dielectric coating of each conductive element, or over the polymer tube, by various methods. For example, the conductive skin can be deposited by vapor deposition, plating, or conductive solution casting or printing. The conductive material is deposited in a layer thin enough to minimize its impact on the flexibility of the lead, which also reduces the impact on surrounding tissue for a long-term implant. The thin layer also enables reduction of the lead diameter to make the implantable lead less invasive to the patient, and reduces the impact of the shielding on MRI quality, e.g., clarity.

A conventional implantable lead includes a multi-conductor cable and, more specifically, a plurality of conductive elements that conduct current between a pulse generator at a proximal end and one or more electrodes at a distal end. The multi-conductor cable typically includes multiple conductive elements individually sheathed, or enclosed, within an electrically insulative material. The multiple conductive elements are then joined, or bundled, within an outer jacket. Generally, the conductive elements are preferably designed with low impedance to enable the conduction of therapeutic current with greater efficiency. A countervailing design objective is to increase impedance of the conductive elements, for example, to better dissipate RF-induced current resulting from electromagnetic field exposure. Alternatively, the cable or individual conductive elements may be shielded using contiguous conductive “foils” or braided wire to reduce or potentially eliminate RF-induced current. Shielding, however, can increase the diameter of the cable and reduce the flexibility, both of which can impact the navigability of the implantable lead and the overall comfort of an implanted lead. Another alternative is to orient and arrange conductive elements within the cable to generate localized canceling current flows.

The impedance of conductive elements can be controlled by adjusting the length of the conductive elements and by changing the materials and construction of the conductive elements. For example, length can be increased by spirally wrapping the conductive elements instead of utilizing a straight length between the pulse generator or sensor device and the distal electrodes. Spiral wrapped conductive elements can have varying pitch to create different levels of impedance. The spiral wrapping also improves durability and can extend the flexure life of the implantable lead. The material properties of conductive elements also dictate the inherent impedance of the conductors. For example, the percentage of silver utilized as a core of each strand of a conductive element can be increased to reduce impedance or decreased to increase impedance. Alternatively, inductors may be added along the length of a conductive element to increase its impedance. Increased impedance resists the conduction of RF-induced current along the length of the implanted lead, dissipating the RF energy as heat that is distributed along the length of the conductive element. The increased impedance also demands more energy from the pulse generator to achieve the same therapeutic effect. The benefits of increased impedance are generally balanced with the disadvantages, for example, increased size of the pulse generator or sensor device, or increased size of battery for the pulse generator.

Localized canceling current flows within a given conductive element can be created by arranging the conductive elements in a particular pattern or geometry. For example, the conductive elements can be overlapped in controlled-length segments over the whole length of the implantable lead. When an electromagnetic field is applied, the induced currents in overlapping segments are oriented in opposing directions, resulting in at least reduced net current. Arranging the conductive elements in this manner also lengthens the conductive elements, resulting in increased impedance. However, the increased length and increased volume of the cable also makes the implantable lead stiffer and limits the ability to minimize the diameter of the lead for the purpose of making the implantable lead less invasive. The increased volume can also reduce the quality of MRI images. More specifically, the increased conductive element volume can reduce clarity in an MRI image by generating a larger disturbance of the RF energy than with a smaller volume lead. Consequently, arranging the conductive elements to generate localized canceling current flows can negatively impact the functionality and cost of the implantable lead, and can negatively impact the MRI itself.

Shielding refers to enclosing the conductive elements within an electrically conductive barrier, or jacket, that absorbs RF energy before it reaches the conductive elements themselves. Shielding is typically composed of a contiguous conductive barrier, e.g., a metallic foil, or composed of a braided conductive barrier. The shield is then bonded to an electrical ground into which the absorbed energy is dissipated. Absent the electrical ground, the absorbed energy would have to dissipate into surrounding tissue, and because the induced current in the shield generally conducts toward either end of the implantable lead, the most significant impact would be on tissue at the distal end and the proximal end. An ideal shield would absorb all RF energy and prevent any RF energy from reaching the conductive elements. However, a typical braided shield absorbs only a portion of the RF energy; the size of that portion depends on how densely the braid is constructed. A contiguous conductive barrier would absorb all RF energy, but such a shield impacts the mechanical properties of the implantable lead, namely the flexibility and the ability to minimize bodily impact and the bodily response to the presence of the implantable lead. A braided shield improves the mechanical properties of the implantable lead over a contiguous shield. For example, the braided shield can improve the durability, the ability to push the lead, and the ability to steer the lead. If the braid is constructed from a relatively high impedance conductor and is densely braided, i.e., gaps among the elements of the braid are minimized, then the portion of RF energy absorbed by the braided shield can be maximized. A high impedance conductor distributes the heat generation induced by the RF energy over the length of the implantable lead. Likewise, the pitch and pick count of the elements of the braid can be adjusted to provide a desired stiffness and the durability while balancing the impact larger voids among the elements has on the portion of RF energy that is absorbed versus reaching the conductive elements. The braided shield can improve steerability by translating torque applied at one end to direct movement at the opposing end without lag or torque buildup. The translation of torque improves the ability to steer the distal end of the implantable lead to a desired location for the therapy. However, the translation of torque can negatively impact the positioning of the implantable lead over time, as bodily motion can result in application of torque to the implantable lead that results in undesired movement of the implantable lead. Consequently, the precise positioning of the electrodes at the distal end of the implantable lead can be disrupted, which reduces the consistency and overall effectiveness of the therapy. Additionally, the overlapping elements inherent to the braided shield results in an increased lead diameter, making the implantable lead more invasive.

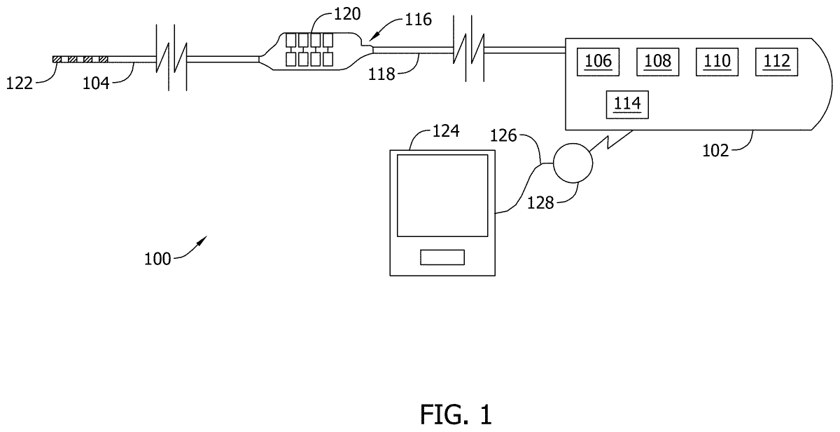

FIG. 1 is a diagram of an example neurostimulation system 100. Neurostimulation system 100 generates electrical pulses for application to tissue of a patient, or subject. Neurostimulation system 100 includes a pulse generator 102 configured to generate the electrical pulses to be delivered to the tissue of the patient via an implantable lead 104. Pulse generator 102 may include an implantable or external pulse generator. Pulse generator 102 generally includes a housing enclosing a controller 106, pulse generation circuits 108, a battery 110, a communication circuit 112, and a memory 114. Pulse generator 102 may also include other appropriate circuits and components to enable operation as described herein. Controller 106 typically includes a microcontroller or other suitable processing device for controlling the various other components of pulse generator 102. Memory 114 stores software code, or computer executable instructions, for execution by controller 106 to control the various other components. Communication circuit 112 may include a wired or wireless interface, such as, for example, serial, Bluetooth, Wi-Fi, or other suitable wired or near-field or far-field wireless communication interface.

Pulse generator 102 may include one or more attached extension components 116, or be connected to one or more separate extension components 116. Alternatively, one or more implantable leads 104 may be connected directly to pulse generator 102. Within pulse generator 102, electrical pulses are generated by pulse generation circuits 108 and are supplied to switching circuitry. The switching circuit connects to output wires, traces, lines, or the like (not shown) that are electrically coupled to internal conductive wires (not shown) of a lead body 118 of extension component 116. The conductive wires are electrically coupled to electrical connectors (e.g., “Bal-Seal” connectors) within a connector portion 120 of extension component 116. The terminals of one or more implantable leads 104 are inserted within connector portion 120 for electrical connection with respective connectors. Pulses generated by pulse generator 102 and conducted through the conductors of lead body 118 are thereby supplied to implantable lead 104. The electrical pulses are then conducted through the conductive elements of implantable lead 104 and applied to tissue of a patient via electrodes 122. Any suitable known or later developed design may be employed for connector portion 120.

For implementation of the components within pulse generator 102, a processor and associated charge control circuitry for an implantable pulse generator is described in U.S. Pat. No. 7,571,007, entitled “SYSTEMS AND METHODS FOR USE IN PULSE GENERATION,” which is incorporated herein by reference. Circuitry for recharging a rechargeable battery of an implantable pulse generator using inductive coupling and external charging circuits are described in U.S. Pat. No. 7,212,110, entitled “IMPLANTABLE DEVICE AND SYSTEM FOR WIRELESS COMMUNICATION,” which is incorporated herein by reference.

An example and discussion of “constant current” pulse generation circuitry is provided in U.S. Patent Publication No. 2006/0170486 entitled “PULSE GENERATOR HAVING AN EFFICIENT FRACTIONAL VOLTAGE CONVERTER AND METHOD OF USE,” which is incorporated herein by reference. One or more sets of such circuitry may be provided within pulse generator 102. Different pulses on different electrodes may be generated using a single set of pulse generation circuits using consecutively generated pulses according to a “multi-stimset program” as is known in the art. Alternatively, multiple sets of such circuitry may be employed to provide pulse patterns that include simultaneously generated and delivered stimulation pulses through various electrodes of one or more stimulation leads as is also known in the art. Various sets of parameters may define the pulse characteristics and pulse timing for the pulses applied to various electrodes as is known in the art. Although constant current pulse generating circuitry is contemplated for some embodiments, any other suitable type of pulse generating circuitry may be employed such as constant voltage pulse generating circuitry.

Implantable lead 104 generally includes a lead body of a plurality of conductive elements within an outer jacket that extend over an axial length from a proximal end of implantable lead 104 to its distal end. The conductive elements electrically couple the plurality of electrodes 122 to a plurality of terminals (not shown) of implantable lead 104. The terminals are adapted to receive electrical pulses and the electrodes 122 are adapted to apply stimulation pulses to tissue of the patient. Also, sensing of physiological signals may occur through electrodes 122, the conductive elements, and the terminals. Additionally or alternatively, various sensors (not shown) may be located near the distal end of implantable lead 104 and electrically coupled to terminals through conductive elements within the lead body 118. Implantable lead 104 may include any suitable number and type of electrodes 122, terminals, and internal conductors.

A controller device 124 may be implemented to recharge battery 110 of pulse generator 102 (although a separate recharging device could alternatively be employed). A “wand” 126 may be electrically connected to controller device 124 through suitable electrical connectors (not shown). The electrical connectors are electrically connected to a coil 128 (i.e., the “primary” coil) at the distal end of wand 126 through respective wires (not shown). Typically, coil 128 is connected to the wires through capacitors (not shown). Also, in some embodiments, wand 126 may comprise one or more temperature sensors for use during charging operations.

The patient then places the primary coil 128 against the patient's body immediately above the secondary coil (not shown), i.e., the coil of the implantable medical device. Preferably, the primary coil 128 and the secondary coil are aligned in a coaxial manner by the patient for efficiency of the coupling between the primary and secondary coils. Controller device 124 generates an AC-signal to drive current through coil 128 of wand 126. Assuming that primary coil 128 and secondary coil are suitably positioned relative to each other, the secondary coil is disposed within the magnetic field generated by the current driven through primary coil 128. Current is then induced by a magnetic field in the secondary coil. The current induced in the coil of the implantable pulse generator is rectified and regulated to recharge the battery of pulse generator 102. The charging circuitry may also communicate status messages to controller device 124 during charging operations using pulse-loading or any other suitable technique. For example, controller device 124 may communicate the coupling status, charging status, charge completion status, etc.

External controller device 124 is also a device that permits the operations of pulse generator 102 to be controlled by a user after pulse generator 102 is implanted within a patient, although in alternative embodiments separate devices are employed for charging and programming. Also, multiple controller devices 124 may be provided for different types of users (e.g., the patient or a clinician). Controller device 124 can be implemented by utilizing a suitable handheld processor-based system that possesses wireless communication capabilities. Software code is stored in memory (not shown) of controller device 124 to control the various operations of controller device 124. Also, the wireless communication functionality of controller device 124 can be integrated within the handheld device package or provided as a separate attachable device. The interface functionality of controller device 124 is implemented using suitable software code for interacting with the user and using the wireless communication capabilities to conduct communications with pulse generator 102.

Controller device 124 preferably provides one or more user interfaces to allow the user to operate pulse generator 102 according to one or more stimulation programs to treat the patient's disorder(s). Each stimulation program may include one or more sets of stimulation parameters including pulse amplitude, pulse width, pulse frequency or inter-pulse period, and pulse repetition parameter (e.g., number of times for a given pulse to be repeated for respective stimset during execution of program), etc. In the methods and systems described herein, stimulation parameters may include, for example, a number of pulses in a burst (e.g., 3, 4, or 5 pulses per burst), an intra-burst frequency (e.g., 500 Hz), an inter-burst frequency (e.g., 40 Hz), and a delay between the pulses in a burst (e.g., less than 1 millisecond (ms)).

Pulse generator 102 modifies its internal parameters in response to the control signals from controller device 124 to vary the stimulation characteristics of stimulation pulses transmitted through implantable lead 104 to the tissue of the patient. Neurostimulation systems, stimsets, and multi-stimset programs are discussed in PCT Publication No. WO 2001/093953, entitled “NEUROMODULATION THERAPY SYSTEM,” and U.S. Pat. No. 7,228,179, entitled “METHOD AND APPARATUS FOR PROVIDING COMPLEX TISSUE STIMULATION PATTERNS,” which are incorporated herein by reference. Example commercially available neurostimulation systems include the EON MINI™ pulse generator and RAPID PROGRAMMER device from Abbott Laboratories.

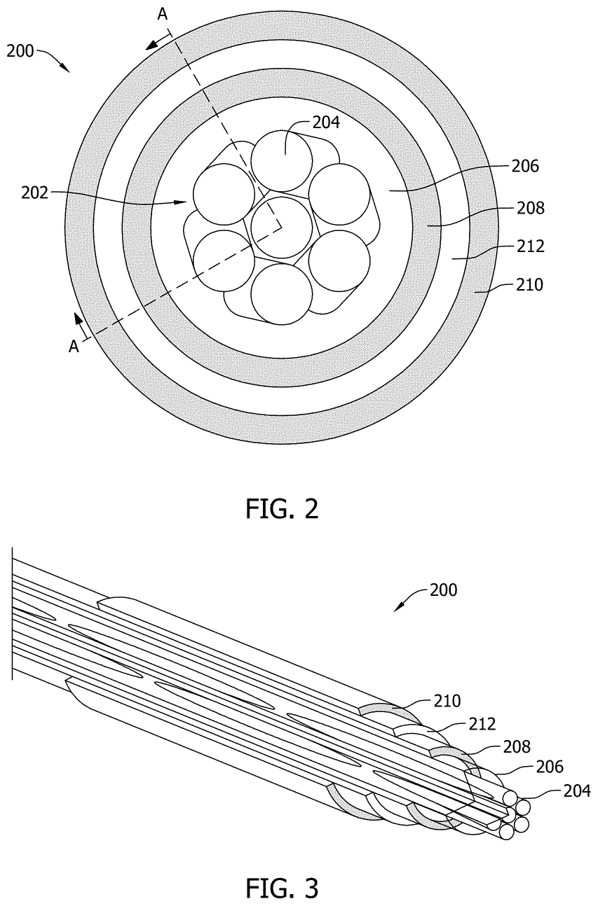

FIG. 2 is a cross-section view of an example conductor assembly 200 for an implantable lead. FIG. 3 is a perspective view of conductor assembly 200 (shown in FIG. 2) with a quarter section A-A removed. Conductor assembly 200 includes a conductive element 202 extending an axial length from a proximal end (e.g., at pulse generator 102) to a distal end (e.g., at electrodes 122). Conductor assembly 200 is composed of seven conductor strands 204. Conductor assembly 200 is illustrated with seven conductor strands 204, although conductive element 202 may be composed of any number of conductor strands 204 for a given implementation. For example, an implantable lead that delivers greater electrical current may require a greater number of conductor strands 204. The seven conductor strands 204 include six conductor strands wrapped, or wound, helically, or spirally, around a seventh core conductor strand 204. In alternate embodiments, strands 204 may be linear conductors that span the length of the implantable lead from proximal end to distal end. Generally, conductive element 202 is designed to optimize efficient therapy and battery life.

Conductor assembly 200 includes an inner dielectric layer 206. Inner dielectric layer 206 coaxially covers, coats, or encloses, conductive element 202 to provide electrical isolation from other conductive elements within the implantable lead, and electrical isolation from electromagnetic shielding. Inner dielectric layer 206 may be composed of any suitable dielectric material, such as, for example, a thermoplastic or thermoset including polymers such as polyimide, ETFE, PFA, PTFE, LCP, or PEEK. Inner dielectric layer 206 is applied in a thin layer to minimize the overall diameter, or profile, of conductor assembly 200 and the implantable lead to reduce the mechanical impact to surrounding tissue and anchor points within the patient. Inner dielectric layer 206 may be applied, for example, by an extrusion process, a dipping process, or an additive manufacturing process, such as deposition.

Conductor assembly 200 includes an inner conductive layer 208 coaxially covering, or enclosing, inner dielectric layer 206 to provide electromagnetic shielding against, for example, RF energy applied during an Mill. Inner conductive layer 208 is a very thin contiguous metal coating applied, for example, by an additive method such as vapor deposition, solution casting, or printing. The very thin layer has a thickness, for example, in the submicron range (less than 1 micron) to over 50 micrometers (microns) and minimizes the dimensional and mechanical impact of the electromagnetic shield on the overall diameter of conductor assembly 200 and the implantable lead. In certain embodiments, the very thin layer has a thickness, for example, in a range of 0.20 thousandths of an inch (mils) to 2.50 mils, or 1 to 50 microns. Conventional conductive shielding (e.g., in coaxial cable) utilizes a wrapped foil or braided wire over a conductive core; however such shielding introduces greater thickness than desired for conductor assembly 200. Inner conductive layer 208 interacts with an electromagnetic field, e.g., from an Mill, resulting in electrical current concentrated on the surface of inner conductive layer 208, i.e., the “skin effect.” Electrical properties of inner conductive layer 208, e.g., the shielding effect, can be optimized to minimize the impact of a specific frequency band of electromagnetic energy, such as RF, on conductive element 202 enclosed within inner conductive layer 208 and inner dielectric layer 206. Optimization of shielding and, more specifically, inner dielectric layer 206 and inner conductive layer 208 may be by material selection, and that material selection may vary over the length of the implantable lead. Inner dielectric layer 206 and inner conductive layer 208 can also be optimized by their respective thickness and their relative thickness.

Conductor assembly 200, in certain embodiments, includes multiple dielectric layers and multiple conductive layers. In alternative embodiments, conductor assembly may include a single conductive layer and a single dielectric layer, e.g., inner dielectric layer 206 and inner conductive layer 208. Referring to FIGS. 2 and 3, conductor assembly 200 includes an outer conductive layer 210 and an outer dielectric layer 212. Outer dielectric layer 212 coaxially covers, or encloses, inner conductive layer 208. Outer dielectric layer 212 is similar to inner dielectric layer 206. In alternative embodiments, outer dielectric layer 212 and inner dielectric layer 206 can be different in composition or in dimension for a given application. Outer dielectric layer 212 provides a dielectric space between inner conductive element 208 and outer conductive element 210. Outer dielectric layer 212 may be composed of any suitable dielectric material, such as, for example, a thermoset, thermoplastic, or polymers including polyimide, ETFE, PFA, PTFA, LCP, or PEEK. Outer dielectric layer 212 is applied in a thin layer to minimize the overall diameter, or profile, of conductor assembly 200 and the implantable lead to reduce the mechanical impact to surrounding tissue and anchor points within the patient. Outer dielectric layer 212 may be applied, for example, by an extrusion process, a dipping process, or an additive manufacturing process, such as deposition.

Outer conductive layer 210 coaxially covers, or encloses, outer dielectric layer 212. Outer conductive layer 210 is similar to inner conductive layer 208. Outer conductive layer 210 provides additional electromagnetic shielding against, for example, RF energy applied during an MRI. Outer conductive layer 210 is a very thin contiguous metal coating applied, for example, by an additive method such as vapor deposition, solution casting, or printing. The very thin layer has a thickness, for example, in the submicron range (e.g., about 500 angstrom) to over 50 microns, and minimizes the dimensional and mechanical impact of the electromagnetic shield on the overall diameter of conductor assembly 200 and the implantable lead. In certain embodiments, the very thin layer has a thickness, for example, in a range of 0.20 mils to 2.50 mils or, alternatively, 1 to 50 microns. Conventional conductive shielding (e.g., in coaxial cable) utilizes a wrapped foil or braided wire over a conductive core; however such shielding introduces greater thickness than desired for conductor assembly 200. Outer conductive layer 210 interacts with an electromagnetic field, e.g., from an MRI, resulting in electrical current concentrated on the surface of outer conductive layer 210.

The shielding effect of the combination of inner conductive layer 208 and outer conductive layer 210 can be optimized to minimize the impact of a specific frequency band of electromagnetic energy, such as RF, on conductive element 202 enclosed within inner conductive layer 208, inner dielectric layer 206, outer conductive layer 210, and outer dielectric layer 212. Optimization of shielding may be by material selection or material thickness. Shielding may be further optimized by varying relative thickness of inner conductive layer 208, outer conductive layer 210, inner dielectric layer 206, and outer dielectric layer 212. Material selections may be the same or different as between inner conductive layer 208 and outer conductive layer 210, or between inner dielectric layer 206 and outer dielectric layer 212.

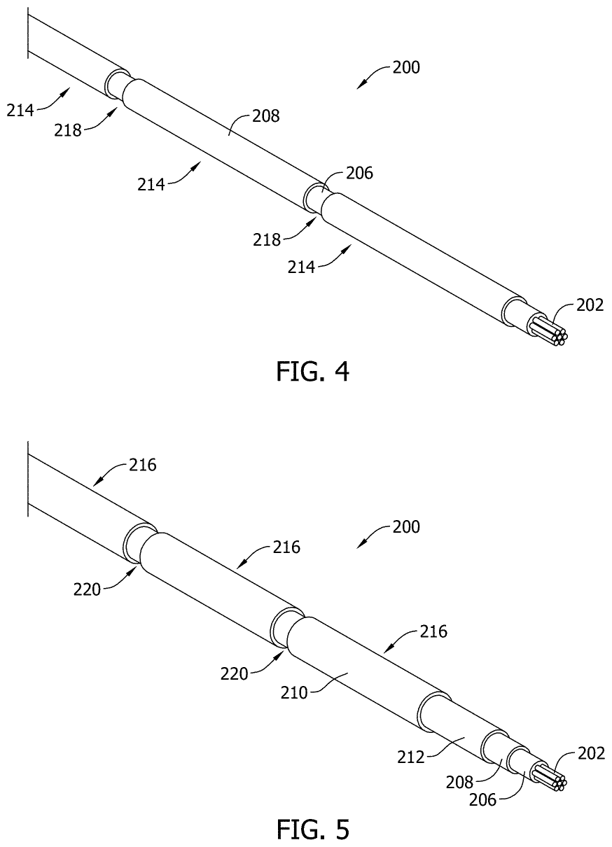

Certain embodiments of conductor assembly 200 include segmented conductive layers to control distribution of energy absorbed by the shield along the length of conductor assembly 200, and to reduce the potential concentration of energy and heating at the proximal and distal ends of the shield conductors. FIG. 4 is a perspective view of conductor assembly 200 including multiple segments 214 of inner conductive layer 208. FIG. 5 is a perspective view of conductor assembly 200 including multiple segments 216 of outer conductive layer 210. The multiple segments 214 of inner conductive layer 208 are at least partially obscured by outer dielectric layer 212 and outer conductive layer 210 in FIG. 5. Each segment 214 and 216 forms an electrically isolated region of inner conductive layer 208 and outer conductive layer 210, respectively. The electrically isolated regions allow energy distribution within a given segment, but induced currents generally remain local to the segment and do not conduct through the length of inner conductive layer 208 or outer conductive layer 210. Segments 214 are separated from each other by a longitudinal dielectric gap 218 that is as small as possible based on manufacturing capabilities. Generally, the smaller dielectric gap 218 is, the less RF energy can reach conductive element 202. The length of each segment 214 can be optimized to provide a desired distribution of energy along the length of conductor assembly 200, and should also be less than one-quarter wavelength of the expected electromagnetic energy induced in inner conductive layer 208, e.g., as a result of an Mill. A length less than one-quarter wavelength reduces the potential energy that may reach, or electromagnetically couple into, conductive element 202. Similarly, segments 216 of outer conductive layer 210 are separated from each other by a longitudinal gap 220 that is as small as possible based on manufacturing capabilities. The length of segments 216 should be less than one-quarter wavelength of the expected electromagnetic energy induced in outer conductive layer 210, e.g., as a result of an MRI. In certain embodiments, as illustrated in FIG. 5, dielectric gaps 218 and 220 are offset longitudinally to further reduce the potential of energy reaching conductive element 202.

Dielectric gaps 218 and 220 are formed in concert with the application of inner conductive layer 208 and outer conductive layer 210. For example, during three-dimensional thin film circuit fabrication processes for forming the conductive layers, or by conductive printing, or by laser ablation. Dielectric gaps 218 and 220, inner conductive layer 208, and outer conductive layer 210 may also be formed using photolithography, wet chemical or plasma etching, or through-mask electroplating. Similarly, inner conductive layer 208 or outer conductive layer 210 may be formed with conductive patterns to further optimize shielding effect and energy dissipation. For example, a conductive layer may be formed with conduction paths in alternating and opposing directions (i.e., “zig-zag”) to produce localized current cancellation within the conductive layer. Such conductive layers with conductive patterns improve upon similar braided shields that generally require greater lengths of wire, increased diameter, and increased stiffness to produce the necessary overlap that can result in localized current canceling. Braided shields also generally are not amenable to segmentation or selective conductive patterns. For example, braided shielding generally requires an overlap of shield wire or shield ribbon during fabrication, and that overlap results in a larger overall diameter once encapsulated, for example, within a polymer jacket. Such braided shielding also impacts the implantable lead's response to torque. For example, the braided shielding can result in a nearly 1-to-1 torque response over the length of an implantable lead, which can improve short term steering and navigation. However, the same implantable lead can create undesired stress and abrasion within a patient when implanted for long durations.

In certain embodiments, outer conductive layer 210 is covered, or enclosed, by a polymer jacket (not shown). A polymer jacket provides shield-to-shield electrical isolation between multiple conductor assemblies 200 within a given implantable lead. For example, one embodiment of an implantable lead may include eight channels, i.e., eight instances of conductor assembly 200. The multiple conductor assemblies 200 may be embedded within the polymer jacket that forms the implantable lead body. Each conductor assembly 200 may be set in a specific place within the polymer jacket, or may be embedded more freely during the fabrication of the polymer jacket. The polymer jacket can also provide thermal insulation and generally enhances the structural integrity of the implantable lead. Because each conductor assembly 200 is individually shielded, such an eight-channel implantable lead is able to shield all conductive elements 202 from RF energy, independent of RF field orientation. Moreover, segments 214 and 216 of inner conductive layer 208 and outer conductive layer 210, respectively, enable localized distribution of absorbed energy along the length of the implantable lead, and reduces the potential for thermal impact at the therapy site.

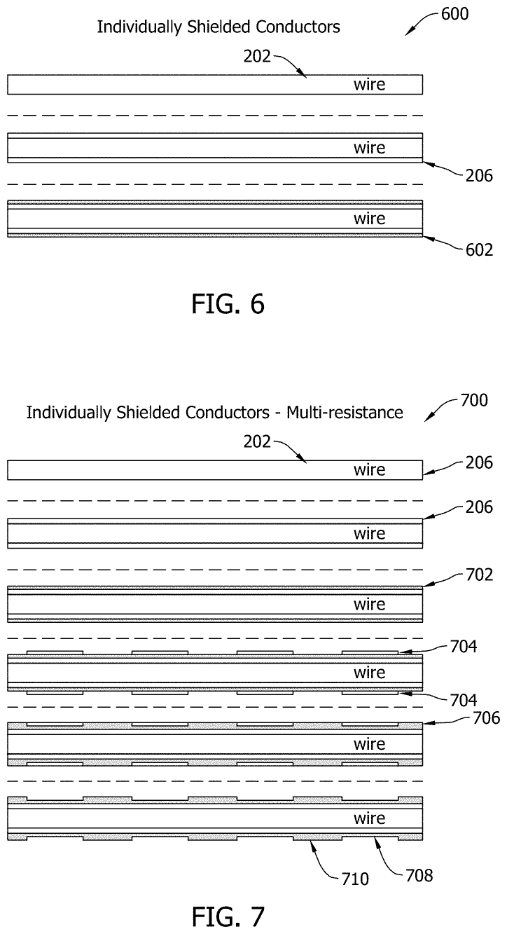

FIG. 6 illustrates fabrication of one embodiment of a conductor assembly 600 having a uniform conductive layer 602. Conductor assembly 600 includes conductive element 202 and inner dielectric layer 206, shown in FIGS. 2-5. Conductive element 202 is composed of, for example, multiple strands of conductors 204, or a single conductor 204. For example, in one embodiment, conductive element 202 has an outer diameter of about 0.003 inches (or 3 mils). In alternative embodiments, conductive element 202 may have an outer diameter of more or less than 0.003 inches to enable a desired amount of therapeutic electrical current, and to also minimize the overall diameter of conductor assembly 600. Conductive element 202 is then covered (i.e., coated or enclosed) with a dielectric material to form inner dielectric layer 206. The dielectric material provides a high-temperature tolerant electrical insulation and is applied in a thickness, for example, of about 0.00025 inches (0.25 mils) to 0.001 inches (1 mil). Conductor assembly 600 is then covered (i.e., coated or enclosed) with a conductive metal layer to form conductive layer 602. Conductive layer 602 is a thin layer having a thickness, for example, in the submicron range to over 50 microns. In certain embodiments, the thin layer has a thickness of about 0.00020 inches (0.20 mils) to 0.001 inches (1 mil). The respective thickness or relative thickness of inner dielectric layer 206 and conductive layer 602 may be optimized to provide a desired shielding effect. Additionally, the specific dielectric material for inner dielectric layer 206 may be selected, for example, from any suitable thermoset, thermoplastic, or other polymer, including materials such as polyimide, ETFE, PTFE, PEEK, LCP, or PFA, among others. Likewise, the specific conductive material for conductive layer 602 may be selected, for example, from any suitable conductive metal, such as gold, niobium, or tantalum, among others.

FIG. 7 illustrates fabrication of one embodiment of a conductor assembly 700 having a conductive shield having a varying resistance over the length of conductor assembly 700. Conductor assembly 700 includes conductive element 202 and inner dielectric layer 206, shown in FIGS. 2-5. Conductive element 202 is composed of, for example, multiple strands of conductors 204, or a single conductor 204. For example, in one embodiment, conductive element 202 has an outer diameter of about 0.003 inches (or 3 mils). In alternative embodiments, conductive element 202 may have an outer diameter of more or less than 0.003 inches to enable a desired amount of therapeutic electrical current, and to also minimize the overall diameter of conductor assembly 200. Conductive element 202 is then covered (i.e., coated or enclosed) with a dielectric material to form inner dielectric layer 206. The dielectric material provides a high-temperature tolerant electrical insulation and is applied in a thickness, for example, of about 0.00025 inches (0.25 mils) to 0.001 inches (1 mil). Conductor assembly 200 is then covered (i.e., coated or enclosed) with a conductive metal layer to form a first conductive layer 702. Conductive layer 702 is a thin layer having a thickness, for example, in the submicron range to over 50 microns. In certain embodiments, the thin layer has a thickness of about 0.00025 inches (0.25 mils) to 0.001 inches (1 mil). A masking 704 is then applied over segments of first conductive layer 702 to form a desired pattern. Masking 704 may include, for example, polyimide, photoresist, or other material that will resist the deposition of additional material in the masked segments. Masking 704 may be applied, for example, by printing or spraying, or other additive procedure to achieve the desired pattern. Alternatively, masking 704 may be applied more generally via a spraying or dipping followed by, for example, a laser ablation procedure to remove masking 704 in areas where additional material will later be deposited. A second conductive layer 706, or through-mask plating, is applied to conductor assembly 700. Second conductive layer 706 is another thin layer of conductive material with a thickness, for example, of about 0.0005 inches (0.5 mils) to 0.0015 inches (1.5 mils). Masking 704 is then stripped away from conductor assembly 700 to reveal segments of first conductive layer 702. Masking 704 may be removed using, for example, a polymer stripping agent or solvent, or by plasma or ultrasonic etching, or any other suitable process for removing the masking material. The combination of first conductive layer 702 and second conductive layer 706 creates a conductive shield having thin regions 708 (e.g., thickness in a range of 0.25 mils to 1.0 mil) and having a corresponding higher electrical resistance, and thick regions 710 (e.g., thickness in a range of 0.75 mils to 2.50 mils) having a corresponding lower electrical resistance.

A similar construction of thin and thick metal segments (e.g., a multi-resistance layer) can be formed by a subtractive process. Subtractive process generally utilizes application of the conductive layer at the final thicker thickness for thick regions 710. Thick regions 710 are masked for protection from, e.g., a wet chemical etch or a plasma dry etch that removes a portion of the conductive material in the unmasked, or thin regions 708. Alternatively, a laser ablation or mechanical machining process can be utilized to thin specific conductive layer regions to make a similar multi-resistance conductive layer. Laser ablation and mechanical machining may enable fabrication without masking or other protective layers.

The respective thickness or relative thickness of inner dielectric layer 206 and conductive layers 702 and 706 may be optimized to provide a desired shielding effect. Additionally, the specific dielectric material for inner dielectric layer 206 may be selected, for example, from any suitable thermoset, thermoplastic, or other polymer, including, for example, polyimide, ETFE, PTFE, PEEK, LCP, or PFA, among others. Likewise, the specific conductive material for conductive layers 702 and 706 may be selected, for example, from any suitable conductive metal, such as gold, niobium, or tantalum, among others.

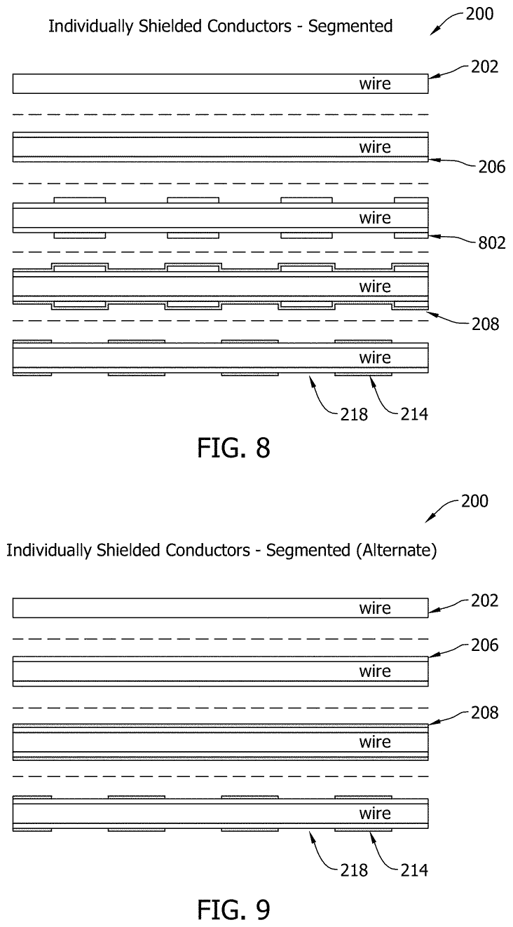

FIG. 8 illustrates one example method of fabrication of one embodiment of conductor assembly 200 having a segmented conductive shielding, shown in FIGS. 2-5. Conductor assembly 200 includes conductive element 202 and inner dielectric layer 206, shown in FIGS. 2-5. Conductive element 202 is composed of, for example, multiple strands of conductors 204, or a single conductor 204. For example, in one embodiment, conductive element 202 has an outer diameter of about 0.003 inches (or 3 mils). In alternative embodiments, conductive element 202 may have an outer diameter of more or less than 0.003 inches to enable a desired amount of therapeutic electrical current, and to also minimize the overall diameter of conductor assembly 200. Conductive element 202 is then covered (i.e., coated or enclosed) with a dielectric material to form inner dielectric layer 206. The dielectric material provides a high-temperature tolerant electrical insulation and is applied in a thickness, for example, of about 0.00025 inches (0.25 mils) to 0.001 inches (1 mil).

A masking 802 is then applied over segments of inner dielectric layer 206 to form a desired pattern. Masking 802 may include, for example, polyimide, photoresist, or other material that will resist the deposition of additional material in the masked segments. Masking 802 may be applied, for example, by printing or spraying, or other additive procedure to achieve the desired pattern. Alternatively, masking 802 may be applied more generally via a spraying, extrusion, or dipping followed by, for example, a laser ablation procedure to remove masking 802 in areas where additional material will later be deposited.

Conductor assembly 200 is then covered (i.e., coated or enclosed) with a conductive metal layer to form inner conductive layer 208 over both inner dielectric layer 206 and masking 802. Inner conductive layer 208 is a thin layer having a thickness, for example, of about 0.00025 inches (0.25 mils) to 0.001 inches (1 mil). The respective thickness or relative thickness of inner dielectric layer 206 and conductive layer 208 may be optimized to provide a desired shielding effect. Additionally, the specific dielectric material for inner dielectric layer 206 may be selected, for example, from any suitable thermoset, thermoplastic, or other polymer, such as polyimide, ETFE, PTFE, PEEK, LCP, or PFA, among others. Likewise, the specific conductive material for conductive layer 208 may be selected, for example, from any suitable conductive metal, such as gold, niobium, or tantalum, among others.

Masking 802 is then stripped away from conductor assembly 200 to reveal segments of inner dielectric layer 206. Masking 802 may be removed using, for example, a polymer stripping agent or solvent, or by plasma or ultrasonic etching, or any other suitable process for removing the masking material. Once masking 802 is removed, inner conductive layer 208 includes numerous segments 214 separated by a dielectric gap 218.

FIG. 9 illustrates another example method of fabrication of one embodiment of conductor assembly 200 having a segmented conductive shielding, shown in FIGS. 2-5. Conductor assembly 200 includes conductive element 202 and inner dielectric layer 206, shown in FIGS. 2-5. Conductive element 202 is composed of, for example, multiple strands of conductors 204, or a single conductor 204. For example, in one embodiment, conductive element 202 has an outer diameter of about 0.003 inches (or 3 mils). In alternative embodiments, conductive element 202 may have an outer diameter of more or less than 0.003 inches to enable a desired amount of therapeutic electrical current, and to also minimize the overall diameter of conductor assembly 200. Conductive element 202 is then covered (i.e., coated or enclosed) with a dielectric material to form inner dielectric layer 206. The dielectric material provides a high-temperature tolerant insulation and is applied in a thickness, for example, of about 0.00025 inches (0.25 mils) to 0.001 inches (1 mil).

Conductor assembly 200 is then covered (i.e., coated or enclosed) with a conductive metal layer to form inner conductive layer 208 over inner dielectric layer 206. Inner conductive layer 208 is a thin layer having a thickness, for example, in the submicron range to over 50 microns. In certain embodiments, inner conductive layer 208 may have a thickness of about 0.00025 inches (0.25 mils) to 0.001 inches (1 mil). The respective thickness or relative thickness of inner dielectric layer 206 and conductive layer 208 may be optimized to provide a desired shielding effect. Additionally, the specific dielectric material for inner dielectric layer 206 may be selected, for example, from any suitable thermoset, thermoplastic, or other polymer, such as polyimide, ETFE, PTFE, PEEK, LCP, or PFA, among others. Likewise, the specific conductive material for conductive layer 208 may be selected, for example, from any suitable conductive metal, such as gold, niobium, or tantalum, among others. Portions of inner conductive layer 208 are then removed to leave segments 214 of conductive material separated by dielectric gaps 218. The conductive material may be removed, for example, by laser ablation to achieve a desired segment length and dielectric gap. Conductive material may alternatively be removed by a combination of masking and chemical or plasma etching. The areas where inner conductive layer 208 remain are masked by a protective layer such as polyimide or photoresist. The un-masked areas are then subjected to wet chemical or dry plasma etching to remove the conductive material and form the dielectric gaps. Finally, the masking material is removed by, for example, a solvent strip, plasma clean, or other similar process.

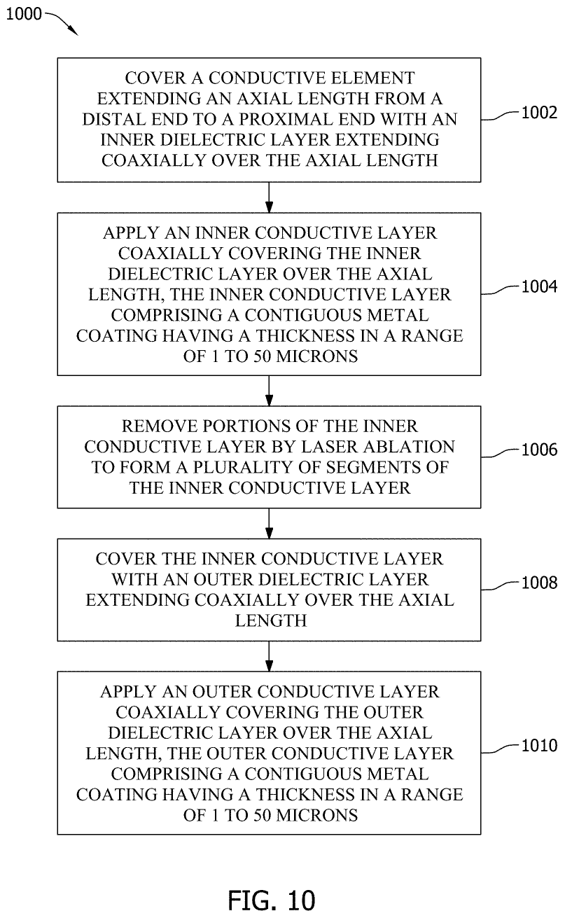

FIG. 10 is a flow diagram of a method 1000 of fabricating a conductor assembly for an implantable lead cable, such as conductor assembly 200 or 600 shown in FIGS. 2-6 and 9. Conductor assembly 200 or 600 includes conductive element 202 and inner dielectric layer 206, shown in FIGS. 2-5. Conductive element 202 is composed of, for example, multiple strands of conductors 204, or a single conductor 204. For example, in one embodiment, conductive element 202 has an outer diameter of about 0.003 inches (or 3 mils). In alternative embodiments, conductive element 202 may have an outer diameter of more or less than 0.003 inches to enable a desired amount of therapeutic electrical current, and to also minimize the overall diameter of conductor assembly 200. Conductive element 202 is then covered 1002 (i.e., coated or enclosed) with a dielectric material to form inner dielectric layer 206. The dielectric material provides a high-temperature tolerant insulation and is applied in a thickness, for example, of about 0.00025 inches (0.25 mils) to 0.001 inches (1 mil). Conductive layer 208 or 602 is then applied 1004 to coaxially cover conductor assembly 200 and, more specifically to cover inner dielectric layer 206 with a thickness in a range of 0.20 mils to 2.50 mils or, alternatively, 1 to 50 microns. Conductive layer 206 or 602 is a thin layer having a thickness, for example, in the submicron range to over 50 microns. In certain embodiments, the thin layer has a thickness of about 0.00020 inches (0.20 mils) to 0.001 inches (1 mil). The respective thickness or relative thickness of inner dielectric layer 206 and conductive layer 206 or 602 may be optimized to provide a desired shielding effect. Additionally, the specific dielectric material for inner dielectric layer 206 may be selected, for example, from any suitable thermoset, thermoplastic, or other polymer, such as polyimide, ETFE, PTFE, PEEK, LCP, or PFA, among others. Likewise, the specific conductive material for conductive layer 206 or 602 may be selected, for example, from any suitable conductive metal, such as gold, niobium, or tantalum, among others. Conductive layer 206 or 602 may be applied, for example, by vapor deposition, solution casting, extrusion, or printing.

In certain embodiments, method 1000 includes removing 1006 portions of inner conductive layer 206 by laser ablation to form a plurality of segments 214 of the inner conductive layer distributed over the axial length and electrically isolated from each other by a plurality of longitudinal dielectric gaps 218 extending axially between each adjacent pair of segments 214. Portions of inner conductive layer 208 may be removed, for example, by laser ablation to achieve a desired segment length and dielectric gap 218.

In certain embodiments, method 1000 includes covering 1008 inner conductive layer 206 with outer dielectric layer 212 extending coaxially over the axial length. Outer conductive layer 210 is then applied 1010 coaxially covering outer dielectric layer 212 over the axial length. Outer conductive layer 210 includes a contiguous metal coating having a thickness in the submicron range to over 50 microns. In certain embodiments, outer conductive layer 210 has a thickness in a range of 0.20 mils to 2.50 mils or, alternatively, 1 to 50 microns.

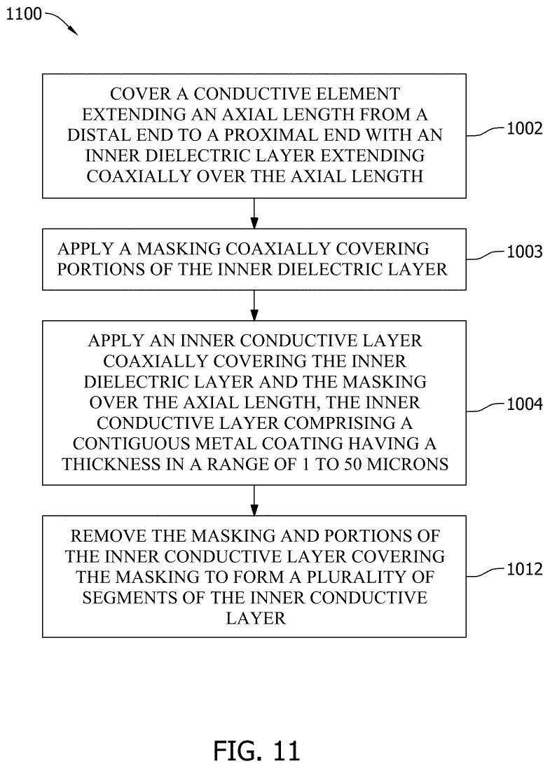

FIG. 11 is a flow diagram of a method 1100 of fabricating a conductor assembly for an implantable lead cable, such as conductor assembly 200 shown in FIGS. 2-5 and 8. Conductor assembly 200 includes conductive element 202 and inner dielectric layer 206, shown in FIGS. 2-5. Conductive element 202 is composed of, for example, multiple strands of conductors 204, or a single conductor 204. For example, in one embodiment, conductive element 202 has an outer diameter of about 0.003 inches (or 3 mils). In alternative embodiments, conductive element 202 may have an outer diameter of more or less than 0.003 inches to enable a desired amount of therapeutic electrical current, and to also minimize the overall diameter of conductor assembly 200. Conductive element 202 is then covered 1002 (i.e., coated or enclosed) with a dielectric material to form inner dielectric layer 206. The dielectric material provides a high-temperature tolerant electrical insulation and is applied in a thickness, for example, of about 0.00025 inches (0.25 mils) to 0.001 inches (1 mil).

A masking 802 is then applied 1003 over segments of inner dielectric layer 206 to form a desired pattern before applying inner conductive layer 208. Masking 802 may include, for example, polyimide or other material that will resist the deposition of additional material in the masked segments. Masking 802 may be applied, for example, by printing or spraying, or other additive procedure to achieve the desired pattern. Alternatively, masking 802 may be applied more generally via a spraying or dipping followed by, for example, a laser ablation procedure to remove masking 802 in areas where additional material will later be deposited.

Conductor assembly 200 is then covered 1004 (i.e., coated or enclosed) with a conductive metal layer to form inner conductive layer 208 over both inner dielectric layer 206 and masking 802. Inner conductive layer 208 is a thin layer having a thickness, for example, of about 0.00025 inches (0.25 mils) to 0.001 inches (1 mil). The respective thickness or relative thickness of inner dielectric layer 206 and conductive layer 208 may be optimized to provide a desired shielding effect. Additionally, the specific dielectric material for inner dielectric layer 206 may be selected, for example, from any suitable thermoset, thermoplastic, or other polymer, such as polyimide, ETFE, PTFE, PEEK, LCP, or PFA, among others. Likewise, the specific conductive material for conductive layer 208 may be selected, for example, from any suitable conductive metal, such as gold, niobium, or tantalum, among others.

Masking 802 is then removed 1012, or stripped away, from conductor assembly 200 to reveal segments of inner dielectric layer 206. Masking 802 may be removed using, for example, a polymer stripping agent or solvent, or by plasma or ultrasonic etching, or any other suitable process for removing the masking material. Once masking 802 is removed, inner conductive layer 208 includes numerous segments 214 separated by a dielectric gap 218.

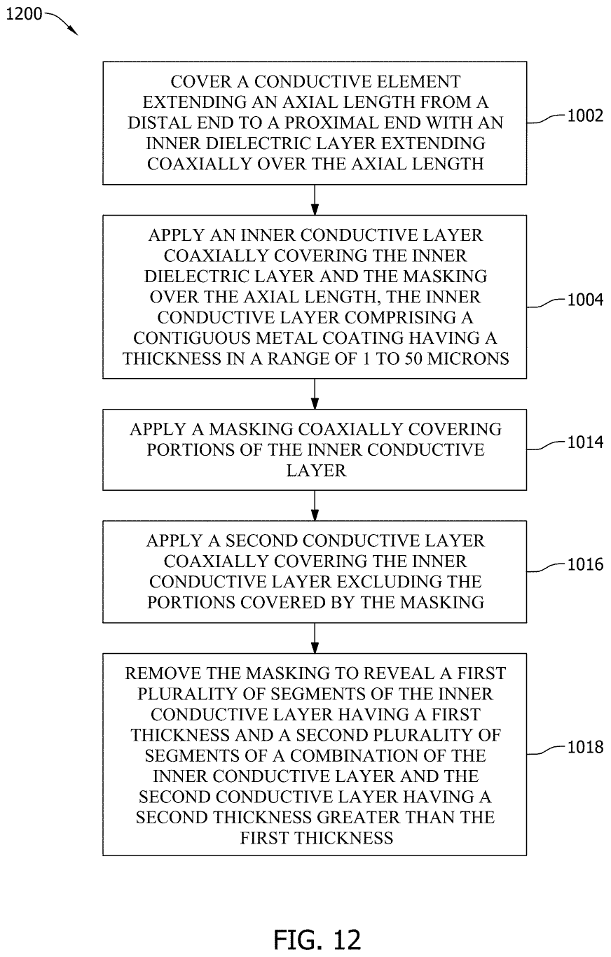

FIG. 12 is a flow diagram of a method 1200 of fabricating a conductor assembly for an implantable lead cable, such as conductor assembly 700 shown in FIGS. 2-5 and 7. Conductor assembly 700 includes conductive element 202 and inner dielectric layer 206, shown in FIGS. 2-5. Conductive element 202 is composed of, for example, multiple strands of conductors 204, or a single conductor 204. For example, in one embodiment, conductive element 202 has an outer diameter of about 0.003 inches (or 3 mils). In alternative embodiments, conductive element 202 may have an outer diameter of more or less than 0.003 inches to enable a desired amount of therapeutic electrical current, and to also minimize the overall diameter of conductor assembly 200. Conductive element 202 is then covered 1002 (i.e., coated or enclosed) with a dielectric material to form inner dielectric layer 206. The dielectric material provides a high-temperature tolerant insulation and is applied in a thickness, for example, of about 0.00025 inches (0.25 mils) to 0.001 inches (1 mil). Conductor assembly 200 is then covered 1004 (i.e., coated or enclosed) with a conductive metal layer to form a first conductive layer 702. Conductive layer 702 is a thin layer having a thickness, for example, in the submicron range to over 50 microns. In certain embodiments, conductive layer 702 has a thickness of about 0.00025 inches (0.25 mils) to 0.001 inches (1 mil). A masking 704 is then applied 1014 over segments of first conductive layer 702 to form a desired pattern. Masking 704 may include, for example, polyimide, photoresist, or other material that will resist the deposition of additional material in the masked segments. Masking 704 may be applied, for example, by printing or spraying, or other additive procedure to achieve the desired pattern. Alternatively, masking 704 may be applied more generally via a spraying or dipping followed by, for example, a laser ablation procedure to remove masking 704 in areas where additional material will later be deposited.

A second conductive layer 706, or through-mask plating, is applied 1016 to conductor assembly 700. Second conductive layer 706 is another thin layer of conductive material with a thickness, for example, in the submicron range to over 50 microns. In certain embodiments, second conductive layer 706 has a thickness of about 0.0005 inches (0.5 mils) to 0.0015 inches (1.5 mils). Masking 704 is then removed 1018, or stripped away, from conductor assembly 700 to reveal segments of first conductive layer 702. Masking 704 may be removed using, for example, a polymer stripping agent or solvent, or by plasma or ultrasonic etching, or any other suitable process for removing the masking material. The combination of first conductive layer 702 and second conductive layer 706 creates a conductive shield having thin regions 708 (e.g., thickness in the submicron range to over 50 microns) and having a corresponding higher electrical resistance, and thick regions 710 (e.g., thickness of the inner conductive layer plus another layer with additional thickness in the submicron range to over 50 microns) having a corresponding lower electrical resistance.

The respective thickness or relative thickness of inner dielectric layer 206 and conductive layers 702 and 706 may be optimized to provide a desired shielding effect. Additionally, the specific dielectric material for inner dielectric layer 206 may be selected, for example, from any suitable thermoset, thermoplastic, or other polymer, such as polyimide, ETFE, PTFE, PEEK, LCP, or PFA, among others. Likewise, the specific conductive material for conductive layers 702 and 706 may be selected, for example, from any suitable conductive metal, such as gold, niobium, or tantalum, among others.

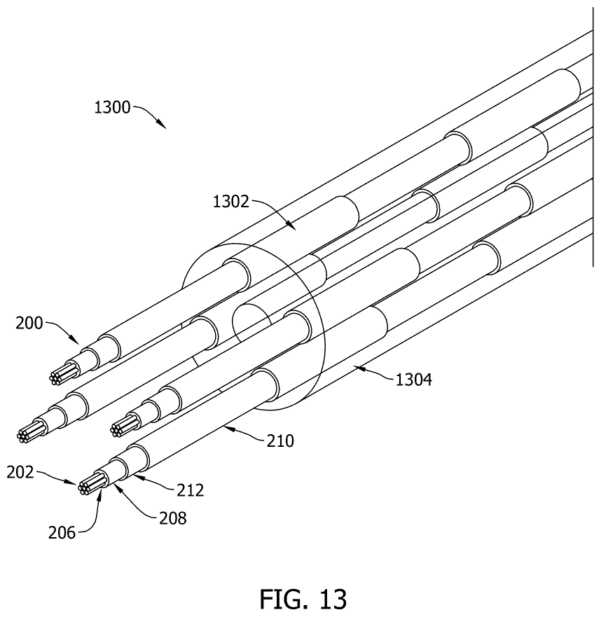

FIG. 13 is a perspective view of an implantable lead body 1300 having a plurality of the conductor assemblies 200 shown in FIGS. 2-5. Implantable lead body 1300 is a multi-conductor lead body having four electrically isolated conductor assemblies 200. In alternative embodiments, implantable lead body 1300 may include one, two, or three conductor assemblies 200, or five or more conductor assemblies 200. Implantable lead body 1300 includes a plurality of lumens 1302 within a polymer tube 1304. Lumens 1302 are pre-formed within polymer tube 1304 to enable greater control and consistency in placement and separation among conductor assemblies 200, and therefore greater control of RF energy dissipation once implanted. In alternative embodiments, each conductor assembly 200 may be coated by a dielectric material individually and then disposed in polymer tube 1304 in a manner that provides electrical isolation between conductor assemblies 200.

Although certain embodiments of this disclosure have been described above with a certain degree of particularity, those skilled in the art could make numerous alterations to the disclosed embodiments without departing from the spirit or scope of this disclosure. All directional references (e.g., upper, lower, upward, downward, left, right, leftward, rightward, top, bottom, above, below, vertical, horizontal, clockwise, and counterclockwise) are only used for identification purposes to aid the reader's understanding of the present disclosure, and do not create limitations, particularly as to the position, orientation, or use of the disclosure. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the spirit of the disclosure as defined in the appended claims.

When introducing elements of the present disclosure or the preferred embodiment(s) thereof, the articles “a,” “an,” “the,” and “said” are intended to mean that there are one or more of the elements. The terms “comprising,” “including,” and “having” are intended to be inclusive and mean that there may be additional elements other than the listed elements.

As various changes could be made in the above constructions without departing from the scope of the disclosure, it is intended that all matter contained in the above description or shown in the accompanying drawings shall be interpreted as illustrative and not in a limiting sense.

What is claimed is:

1. A conductor assembly for an implantable lead cable, the conductor assembly comprising:
a conductive element extending over an axial length from a proximal end to a distal end;
an inner dielectric layer coaxially covering the conductive element over the axial length; and
an inner conductive layer coaxially covering the inner dielectric layer over the axial length, the inner conductive layer comprising a contiguous metal coating having a thickness in a range of 1 to 50 microns.

2. The conductor assembly of claim 1, wherein the conductive element comprises a plurality of conductor strands extending over the axial length.

3. The conductor assembly of claim 2, wherein the plurality of conductor strands comprise a core strand extending linearly over the axial length, and a plurality of helical strands wound around the core strand.

4. The conductor assembly of claim 2, wherein the plurality of conductor strands extend linearly over the axial length.

5. The conductor assembly of claim 1, wherein the inner dielectric layer comprises a thermoplastic material.

6. The conductor assembly of claim 1, wherein the inner dielectric layer is applied with a thickness in a range of 0.20 mils to 1.20 mils.

7. The conductor assembly of claim 1, wherein the inner conductive layer comprises a plurality of segments distributed over the axial length and electrically isolated from each other by a plurality of longitudinal dielectric gaps extending axially between each adjacent pair of segments.

8. The conductor assembly of claim 7, wherein the longitudinal dielectric gap has an axial dimension of less than 0.010 inches.

9. The conductor assembly of claim 1, wherein the inner conductive layer comprises gold.

10. The conductor assembly of claim 1, wherein the inner conductive layer comprises a first plurality of segments having a first thickness and a second plurality of segments having a second thickness greater than the first thickness, and wherein the first plurality of segments and the second plurality of segments are distributed over the axial length.

11. The conductor assembly of claim 1 further comprising:
an outer dielectric layer coaxially covering the inner conductive layer over the axial length; and
an outer conductive layer coaxially covering the outer dielectric layer over the axial length, the outer conductive layer comprising a contiguous metal coating having a thickness in a range of 1 to 50 microns.

12. The conductor assembly of claim 11, wherein the inner conductive layer comprises a first plurality of segments distributed over the axial length and electrically isolated from each other by a first plurality of longitudinal dielectric gaps extending axially between each adjacent pair of segments, wherein the outer conductive layer comprises a second plurality of segments distributed over the axial length and electrically isolated from each other by a second plurality of longitudinal dielectric gaps extending axially between each adjacent pair of segments, and wherein the first plurality of segments of the inner conductive layer are distributed relative to the second plurality of segments of the outer conductive layer such that the first plurality of longitudinal dielectric gaps are offset axially from the second plurality of longitudinal dielectric gaps.

13. A method of fabricating a conductor assembly for an implantable lead cable, the method comprising:
covering a conductive element extending an axial length from a distal end to a proximal end with an inner dielectric layer extending coaxially over the axial length; and
applying an inner conductive layer coaxially covering the inner dielectric layer over the axial length, the inner conductive layer comprising a contiguous metal coating having a thickness in a range of 1 to 50 microns.

14. The method of claim 13, wherein applying the inner conductive layer comprises applying the contiguous metal coating by vapor deposition.

15. The method of claim 13, wherein applying the inner conductive layer comprises applying the contiguous metal coating by solution casting.

16. The method of claim 13, wherein applying the inner conductive layer comprises applying the contiguous metal coating by printing.

17. The method of claim 13 further comprising removing portions of the inner conductive layer by laser ablation to form a plurality of segments of the inner conductive layer distributed over the axial length and electrically isolated from each other by a plurality of longitudinal dielectric gaps extending axially between each adjacent pair of segments.

18. The method of claim 13 further comprising:
applying a masking, before applying the inner conductive layer, coaxially covering portions of the inner dielectric layer; and
removing, after applying the inner conductive layer coaxially covering the inner dielectric layer and the masking, the masking and portions of the inner conductive layer covering the masking to form a plurality of segments of the inner conductive layer distributed over the axial length and electrically isolated from each other by a plurality of longitudinal dielectric gaps extending axially between each adjacent pair of segments.

19. The method of claim 13 further comprising:
applying a masking coaxially covering portions of the inner conductive layer;
applying a second conductive layer coaxially covering the inner conductive layer excluding the portions covered by the masking; and
removing the masking to reveal a first plurality of segments of the inner conductive layer having a first thickness and a second plurality of segments of a combination of the inner conductive layer and the second conductive layer having a second thickness greater than the first thickness, and wherein the first plurality of segments and the second plurality of segments are distributed over the axial length.

20. The method of claim 13 further comprising:
covering the inner conductive layer with an outer dielectric layer extending coaxially over the axial length; and
applying an outer conductive layer coaxially covering the outer dielectric layer over the axial length, the outer conductive layer comprising a contiguous metal coating having a thickness in a range of 1 to 50 microns.

21. The method of claim 20, wherein applying the outer conductive layer comprises forming a plurality of segments distributed over the axial length and electrically isolated from each other by a plurality of longitudinal dielectric gaps extending axially between each adjacent pair of segments.

22. The method of claim 20, wherein applying the inner conductive layer comprises forming a first plurality of segments distributed over the axial length and electrically isolated from each other by a first plurality of longitudinal dielectric gaps extending axially between each adjacent pair of segments, wherein applying the outer conductive layer comprises forming a second plurality of segments distributed over the axial length and electrically isolated from each other by a second plurality of longitudinal dielectric gaps extending axially between each adjacent pair of segments, and wherein the first plurality of segments of the inner conductive layer are distributed relative to the second plurality of segments of the outer conductive layer such that the first plurality of longitudinal dielectric gaps are offset axially from the second plurality of longitudinal dielectric gaps.

23. The method of claim 13, wherein applying the inner conductive layer comprises applying the contiguous metal coating by extruding both the inner dielectric layer and inner conductive layer simultaneously over the conductive element.

24. An implantable lead comprising:
at least one electrode disposed at a distal end opposite a proximal end configured to be coupled to a pulse generator;
a plurality of conductor assemblies extending over an axial length from the proximal end to the at least one electrode at the distal end, each conductor assembly comprising:
a conductive element extending over the axial length;
an inner dielectric layer coaxially covering the conductive element over the axial length; and
an inner conductive layer coaxially covering the inner dielectric layer over the axial length, the inner conductive layer comprising a contiguous metal coating having a thickness in a range of 1 to 50 microns; and
a polymer jacket coaxially covering the plurality of conductor assemblies over the axial length, the polymer jacket electrically isolating respective inner conductive layers of the plurality of conductor assemblies.
